(19) United States
(12) Patent Application Publication
(10) Pub. No.: US 2018/0310327 A1
(43) Pub. Date: Oct. 25, 2018

(54) WIRELESS PATIENT MONITORING SYSTEM AND METHOD WITH IMPROVED PHYSIOLOGICAL DATA TRANSMISSION

(71) Applicant: General Electric Company, Schenectady, NY (US)

(72) Inventors: Lauri Tapio Aarnio, Helsinki (FI); Juha Virtanen, Helsinki (FI)

(73) Assignee: General Electric Company, Schenectady, NY (US)

(21) Appl. No.: 15/491,647

(22) Filed: Apr. 19, 2017

(51) Int. Cl. H04W 72/12; H04W 28/06; A61B 5/00; A61B 5/0452; A61B 5/145; A61B 5/0476

(52) U.S. Cl. CPC H04W 72/1236; H04W 28/065; A61B 5/7225; A61B 5/0022; A61B 5/0452; A61B 5/0006; A61B 5/0476; A61B 5/742; A61B 5/0028; A61B 5/002; A61B 5/14546

(57) ABSTRACT

A wireless patient monitoring system includes a sensing device having a sensor that senses a physiological signal of a patient, an analog-to-digital converter that generates a stream of digitized signal samples based on the physiological signal, and a first processor. Each sensing device further includes a transmission management module executable on the first processor to divide the stream of digitized signal samples into two or more interlaced subsets containing non-adjacent signal samples from the stream of digitized signal samples, generate at least one subset packet based on each of the two or more interlaced subsets, and control wireless transmission of the subset packets. The system further includes a receipt management module executable on a second processor to receive the subset packets, extract each of the two or more interlaced subsets of non-adjacent signal samples, and piece the non-adjacent signal samples together to reconstruct the stream of digitized signal samples.

### BACKGROUND

The present disclosure relates generally to patient monitoring devices and systems for monitoring a patient's physiology and health status. More specifically, the present disclosure relates to patient monitoring devices, systems, and methods that wirelessly transmit patient physiological data.

In the field of medicine, physicians often desire to monitor multiple physiological characteristics of their patients. Oftentimes, patient monitoring involves the use of several separate monitoring devices simultaneously, such as an electrocardiograph (ECG), a pulse oximeter, an electroencephalograph (EEG), etc. Several separate patient monitoring devices are often connected to a patient, tethering the patient to multiple bulky bedside devices via physical wiring or cables. Multi-parameter monitors are also available where different sensor sets may be connected to a single monitor. However, such multi-parameter systems may be even more restrictive than separate monitoring devices because they require all of the sensors attached to a patient to be physically attached to a single monitor, resulting in multiple wires running across the patient's body. Thus, currently available patient monitoring devices often inhibit patient movement, requiring a patient to stay in one location or to transport a large monitor with them when they move from one place to another.

Further, currently available monitoring devices are often power intensive and either require being plugged in to a wall outlet or require large battery units that have to be replaced and recharged every few hours. Thus, monitoring multiple patient parameters is power intensive and battery replacement is costly in labor and parts. Thus, frequent monitoring is often avoided in order to limit cost and patient discomfort, and instead patient parameters are infrequently spot checked, such as by periodic nurse visits one or a few times a day. However, patients that are not being regularly monitored may encounter risky health situations that that go undetected for a period of time, such as where rapid changes occur in physiological parameters that are not checked by a clinician until hours later or until a critical situation occurs. Thus, it is often desirable to continually or frequently obtain certain physiological information from a patient, which is a battery-intensive endeavor.

### SUMMARY

This Summary is provided to introduce a selection of concepts that are further described below in the Detailed Description. This Summary is not intended to identify key or essential features of the claimed subject matter, nor is it intended to be used as an aid in limiting the scope of the claimed subject matter.

In one embodiment, a wireless patient monitoring system includes a sensing device having a sensor that senses a physiological signal of a patient, an analog-to-digital converter that generates a stream of digitized signal samples based on the physiological signal, and a first processor. Each sensing device further includes a transmission management module executable on the first processor to divide the stream of digitized signal samples into two or more interlaced subsets containing non-adjacent signal samples from the stream of digitized signal samples, generate at least one subset packet based on each of the two or more interlaced subsets, and control wireless transmission of the subset packets. The system further includes a receipt management module executable on a second processor to receive the subset packets, extract each of the two or more interlaced subsets of non-adjacent signal samples, and piece the non-adjacent signal samples together to reconstruct the stream of digitized signal samples.

One embodiment of a method of patient physiological monitoring includes sensing at least one physiological signal of the patient, generating a stream of digitized signal samples based on the physiological signal, and dividing a time section of the stream of digitized signal samples into two or more interlaced subsets, wherein each interlaced subset contains non-adjacent signal samples from the times section of the stream of digitized signal samples. A subset packet is generated based on each of the two or more interlaced subsets, and then each subset packet is transmitted. Each of the subset packets is received and the interlaced subsets are extracted. The non-adjacent signal samples in the interlaced subsets are then pieced together to reconstruct the time section of the stream of digitized signal samples.

Various other features, objects, and advantages of the invention will be made apparent from the following description taken together with the drawings.

### BRIEF DESCRIPTION OF THE DRAWINGS

The present disclosure is described with reference to the following Figures.

FIG. 1 provides a schematic diagram of one embodiment of a wireless patient monitoring system.

FIG. 2 depicts another embodiment of a wireless patient monitoring system.

FIG. 3 depicts a computing system portion of an exemplary wireless patient monitoring system of the present disclosure.

FIG. 4 depicts a simplified ECG recording of two heart beats and exemplary data packet sizes for the transmission of the exemplary ECG data using prior art systems and methods.

FIG. 5 depicts an exemplary ECG recording containing 1 second of data capturing one heart beat, represented by 600 samples.

FIG. 6 provides a graphical representation of the 600 samples of the signal depicted in FIG. 5 divided into five interlaced subsets.

FIGS. 7a-7d depict exemplary embodiments of four subset packets generated based on the interlaced subsets depicted at FIG. 6.

FIG. 8 graphically depicts one embodiment where a transmission start time for each subsequent subset packet is delayed such that the transmission of each subset packet is started at a different time.

FIG. 9 depicts another embodiment where each of three physiological signals recorded from a patient is divided into three subset packets, where the interlaced subsets for each channel are staggered with respect to their sample start point.

FIGS. 10-11 depict exemplary methods of monitoring patient physiology according to the present disclosure.

### DETAILED DESCRIPTION

The present inventor has recognized that wireless monitoring systems are desirable, for example to provide more comfort and mobility to the patient being monitored. The patient's movement is not inhibited by wires between sensor devices and/or computing devices that collect and process the physiological data from the patient. Thus, small sensing devices and sensors that can be easily attached to the patient's body are desirable, such as sensing devices that are wearable portable computing devices. In order to do so, the size of the wireless sensing devices must be small. The present inventor has recognized that an important aspect of decreasing the size and weight of wireless sensing devices is decreasing battery size, and that a weakness in the development of wireless sensing devices has been power consumption and requirement for long battery times.

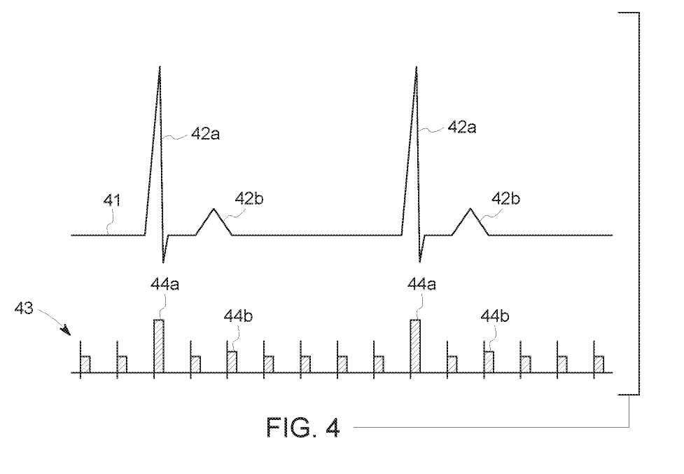

The inventor has recognized that data transmission plays a significant role in battery consumption and that data compression is helpful, or even necessary, for reducing the amount of battery power consumed by data transmission. This is especially the case for sensing devices that provide a continuous stream of measurement results, such as ECG, SPO2, EEG, and the like. However, the inventor has recognized that one problem with using standard data compression algorithms in certain physiological monitoring applications is that the packet size of the compressed data can reveal information about the compressed data, and thus reveal patient health information, which is undesirable from a patient-confidentiality standpoint. FIG. 4 exemplifies this problem with respect to ECG data. FIG. 4 shows a simplified ECG signal 41 of a patient over two heart beats, and thus including two QRS complexes. Represented below is a series of corresponding compressed data packets 43 containing the compressed signal samples of the ECG signal 41.

As demonstrated, the compressed data packets 43 containing data representing areas of significant change in the physiological signal 41 are larger than those where the physiological signal 41 remains relatively constant. Thus, each QRS wave 42a section of the physiological signal 41 represents the largest change between each signal sample and generates the largest compressed data packet 44a. The T wave 42b of the ECG signal represents the second largest change magnitude between each signal sample, and thus generates the second largest data packet 44b. Accordingly, the patient heart rate and relative magnitude of the QRS waves and T waves can be obtained by simply looking at the size of the data packets 43 themselves, without access to the actual ECG data contained in the data packets 43. The inventor has recognized that this poses a patient confidentiality concern because certain information can be obtained about the patient's physiology by simply observing the data packet size and without actually obtaining the data contained in the packets (which is presumably encrypted). This is highly undesirable from a patient-confidentiality standpoint.

While this problem could be solved by not compressing the data, the inventor recognized that data compression is highly valuable or even necessary in wireless monitoring applications involving transmission of continuous measurement results in order to meet battery utilization constraints.

Further, the inventor has recognized that serial transmission of data packets as depicted in FIG. 4 is unreliable, as certain packets contain most or all of the valuable information, especially in an ECG application, and thus loss of one or two data packets due to a momentary data transmission interruption an be highly problematic. For example, if packet 44a is interrupted, then the entire QRS waveform is lost and the remaining data for that heart beat is largely irrelevant and unhelpful.

In view of the foregoing challenges and problems regarding transmission of a continuous physiological data stream, the inventor developed the systems and methods disclosed herein. Namely, instead of sending digitized samples in a strict first in/first order and compressing sequential samples into sequential data packets, the inventor developed the presently disclosed systems and methods wherein sequential samples are reorganized into several interlaced subsets which are then encrypted and transmitted. To provide just one example, a contiguous set of digitized samples from a data stream may be divided into three interlaced subsets, where the first interlaced subset contains the first, fourth, seventh, . . . sample; the second subset contains the second, fifth, eighth, . . . sample; and the third interlaced subset contains the third, sixth, ninth, . . . sample. Each interlaced subset is then packetized into a subset packet and transmitted separately.

Use of this disclosed method eliminates the problem explained with respect to FIG. 4 of the packet size revealing information regarding the physiological signal. Further masking any connection between packet size and information contained in the physiological data may be provided in some embodiments by delaying transmission of one or more of the packets with respect to the other packets. Alternatively or additionally, further masking may be provided by staggering the start points for each interlaced subset (first signal sample in the respective pattern portion).

Any number of various algorithms or patterns may be used to create the interlaced subsets, and the data is simply reorganized accordingly upon receipt at a wireless receiver in order to reconstruct the digitized physiological signal on the receiving end. Likewise, each interlaced subset can be compressed, such as using any standard compression algorithm. Thus, data compression can be utilized to minimize battery consumption by the data transmission without concern regarding packet size and patient confidentiality.

Moreover, this transmission method and system increases data reliability since each data packet contains noncontiguous samples. Accordingly, the loss of one or two packets, such as due to interference or collisions during transmission, does not result in the loss of the physiological signal entirely, only the loss of some non-contiguous samples in the dataset (and thus a possible reduction in resolution rather than a total loss of the signal section). Accordingly, the system and method disclosed herein offers increased reliability and resiliency of the system, for example, increasing system tolerance to collisions during transmission. The disclosed the system and method further offers increased security of patient physiological information while also minimizing the power consumption by wireless sensing devices. Furthermore, the disclosed transmission steps can be executed in a way to avoid significant and undo delays in the time between sensing the data and displaying the data to a clinician by displaying an estimate of the physiological signal based on an initial interlaced subset, and then updating the display as additional interlaced subsets are received.

In various depicted embodiments, wireless sensing devices measuring different physiological parameters may be networked to a central hub or primary sensing device that communicates with a central, host network, such as of the medical facility. In another embodiment, the wireless sensing devices may communicate with the host network that calculates the patient stability index and assigns the measurement intervals. There, the wireless sensing devices may communicate with the host network directly, or indirectly through the hub. For example the hub may serve as an amplifier and/or router for communication between the wireless sensing devices and the host network. The data transmission methods and systems described herein may be utilized for transmission of patient data by and between one or more of the sensing devices hub, and/or host network.

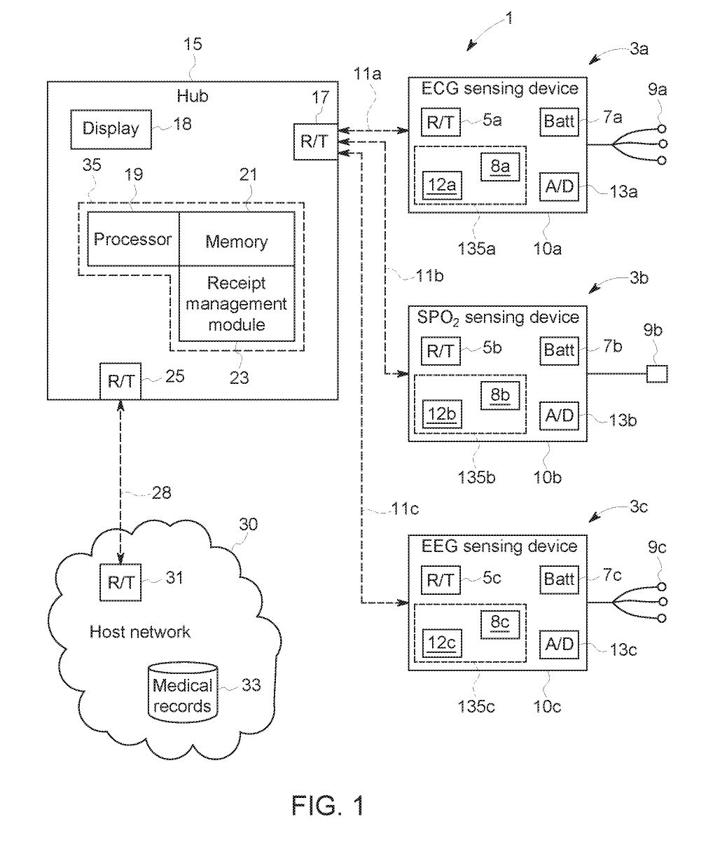

FIG. 1 depicts one embodiment of a patient monitoring system 1 containing three wireless sensing devices 3a-3c in wireless communication with a hub 15. The hub 15 is in wireless communication with a host network 30 that contains medical records database 33. For example, the hub device 15 may be attached to the patient's body, placed on or near the patient's bed, or positioned within range of the patient, such as in the same room as the patient. The hub 15 may be a separate, stand alone device, or it may be incorporated and/or housed with another device within the system 1, such as housed with one of the wireless sensing devices 3a-3c. Each wireless sensing device 3a-3c contains one or more sensors 9a-9c for measuring a physiological parameter from a patient, and also includes a base unit 10a-10c that receives the physiological parameter measurements from the sensors 9a-9c and transmits a parameter dataset based on those measurements to the hub device 15 via communication link 11a-11c. The sensors 9a-9c may be connected to the respective base unit 10a-10c by wired or wireless means. The sensors 9a-9c may be any sensors, leads, or other devices available in the art for sensing or detecting physiological information from a patient, which may include but are not limited to electrodes, lead wires, or available physiological measurement devices such as pressure sensors, flow sensors, temperature sensors, blood pressure cuffs, pulse oximetry sensors, or the like.

In the depicted embodiment, a first wireless sensing device 3a is an ECG sensing device having sensors 9a that are ECG electrodes. A second wireless sensing device 3b is a peripheral oxygen saturation (SpO2) monitor having sensor 9b that is a pulse oximetry sensor, such as a standard pulse oximetry sensor configured for placement on a patient's fingertip. A third wireless sensing device 3c is an EEG monitor having sensors 9c that are EEG electrodes. It should be understood that the patient monitoring system 1 of the present disclosure is not limited to the examples of sensor devices provided, but may be configured and employed to sense and monitor any clinical parameter. The examples provided herein are for the purposes of demonstrating the invention and should not be considered limiting.

The base units 10a-10c of each of the exemplary wireless sensing devices 3a-3c include analog-to-digital (A/D) converters 13a-13c, which may be any devices or logic sets capable of digitizing analog physiological signals recorded by the associated sensors 9a-9c. For example, the A/D converters 13a-13c may be Analog Front End (AFE) devices. The base units 10a-10c may further include processors 12a-12c that receive the digital physiological data from the A/D converters 13a-13c and create a stream of physiological data that gets transmitted and/or for the host network 30. Each base unit 10a-10c may be configured differently depending on the type of wireless sensing device, and may be configured to perform various signal processing functions and or sensor control functions. To provide just a few examples, the processor 12a in the ECG sensing device 3a may be configured to filter the digital signal from the ECG sensors 9a to remove artifact and/or to perform various calculations and determinations based on the recorded cardiac data, such as heart rate, QRS interval, ST-T interval, or the like. Each wireless sensing device 3a-3c includes a battery 7a-7c that stores energy and powers the various aspects thereof. Each processor 12a-12c may further include power management capabilities, especially where the respective wireless sensing device 3a-3c contains more demanding electromechanical aspects.

In other embodiments, the processors 12a-12c may not perform any signal processing tasks and may simply be configured to perform necessary control functions for the respective wireless sensing device 3a-3c. In such an embodiment, the data to be transmitted only includes the digitized raw data or digitized filtered data from the various sensor devices 9a-9c.

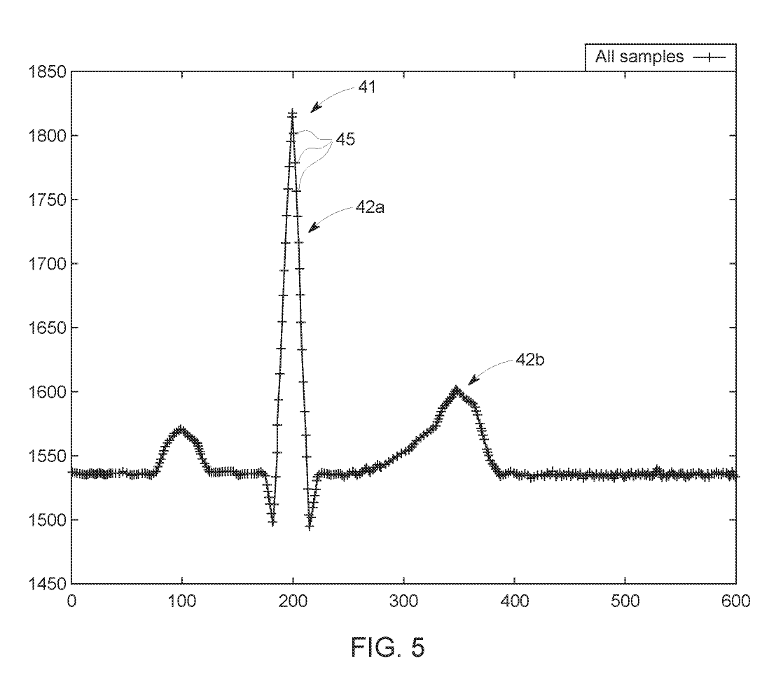

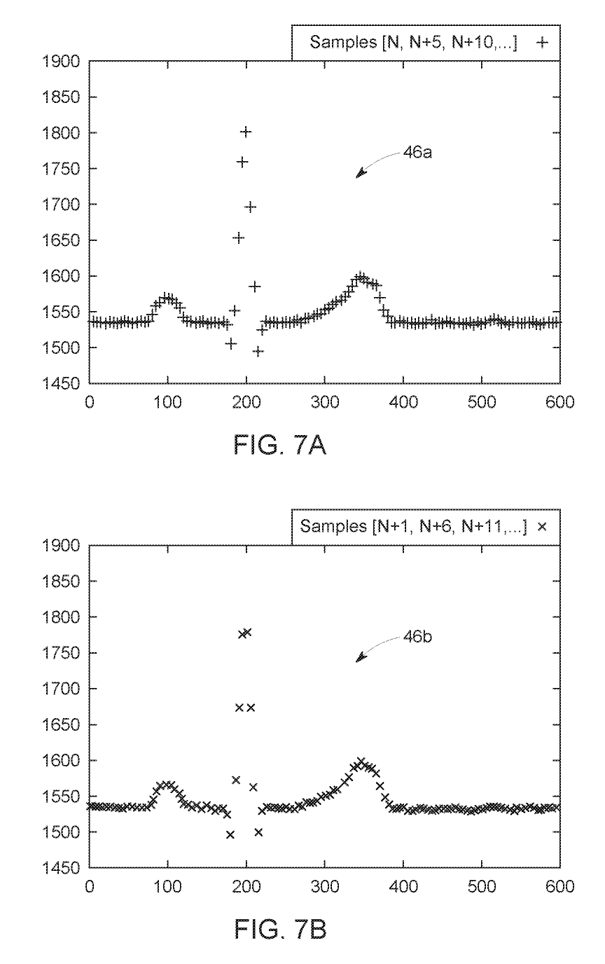

The data is then transmitted according to the methods described herein. For example, each sensing device 3a-3c may contain a transmission management module 8a-8c that is a set of software instructions executable within the computing system 135a-315c of the respective sensing device to divide a set of digitized signal samples into two or more interlaced subsets, wherein each interlaced subset contains non-adjacent signal samples from the stream of digitized samples. A subset packet is then generated for each of the two or more interlaced subsets, and each subset packet is transmitted, such as to the hub 15 and/or to the host network 30. This process is described in more detail and exemplified below with respect to FIGS. 5-9 and the associated description.

The subset packets transmitted by the respective sensing devices 3a-3c are received at a receiving device, such as hub 15 and/or host network 30 where they are processed to extract the interlaced subsets of signal samples from each subset packet, and then to piece the non-adjacent signal samples from the interlaced subsets together in order to reconstruct the stream of digitized signal samples. In the embodiment of FIG. 1, the receipt and reconstruction steps are executed by the receipt management module 23 in the hub 15. Specifically, the receipt management module 23 may be executable on a processor 19 within the hub 5 in order to receive subset packets from one or more of the sensing devices 3a-3c and to extract the interlaced subsets of non-adjacent signal samples therefrom. The receipt management module 23 is aware of the interlacing pattern(s) used to create the interlaced subsets, and thus pieces together the non-adjacent signal samples in the interlaced subsets accordingly in order to reconstruct the stream of digitized signal samples. The receipt management module 23 may further execute steps to display a graphical representation of the physiological signal on a display 18 on or associated with the hub 15.

For transmitting the subset packets, the receiver/transmitter 5a-5c of each wireless sensing device 3a-3c communicates via the respective communication link 11a-11c with the receiver/transmitter 17 of the hub 15, which may include separate receiving and transmitting devices or may include an integrated device providing both functions, such as a transceiver. The receiver/transmitters 5a-5c of the wireless sensing devices 3a-3c and the receiver/transmitter 17 of the hub 15 may be any radio frequency devices known in the art for wirelessly transmitting data between two points according to any of numerous communication standards. In one embodiment, the receiver/transmitters 5a-5c and 17 may be body area network (BAN) devices, such as medical body area network (MBAN) devices, that operate as a wireless network. For example, the wireless sensing devices 3a-3c may be wearable or portable computing devices in communication with a hub 15 positioned in proximity of the patient. Other examples of radio protocols that could be used for this purpose include, but are not limited to, Wi-Fi, Bluetooth, Bluetooth Low Energy (BLE), ANT, IEEE 802.15.4 (e.g., ZIGBEE or 6LoWPAN).

The hub 15 may then communicate with a host network 30 via a wireless communication link 28, such as to transmit the data from the respective wireless sensing devices 3a-3c to the host network 30 for display and/or for storage in the patient's medical record. The hub 15 has receiver/transmitter 25 that communicates with a receiver/transmitter 31 associated with the host network 30 on communication link 28, which may operate according to a network protocol appropriate for longer-range wireless transmissions, such as on the wireless medical telemetry service (WMTS) spectrum or on a Wi-Fi-compliant wireless local area network (LAN). The host network 30 may be, for example, a local computer network having servers housed within a medical facility treating the patient, or it may be a cloud-based system hosted by a cloud computing provider. The host network 30 may include a medical records database 33 housing the medical records for the patient, which may be updated to store the parameter datasets recorded and transmitted by the various wireless sensing devices 3a-3c. The host network 30 may further include other patient care databases, such as for monitoring, assessing, and storing particular patient monitoring data. For example, the host network may include an ECG database, such as the MUSE ECG management system produced by General Electric Company of Schenectady, N.Y.

In certain embodiments the hub 15 may transmit or relay the subset packets to the host network 30, which may also contain a receipt management module 23 that extracts and pieces together the non-adjacent signal samples in the interlaced subsets. In other embodiments, the hub 15 may transmit the data by other means and in other forms, such as directly transmitting the uncompressed stream of digitized signal samples.

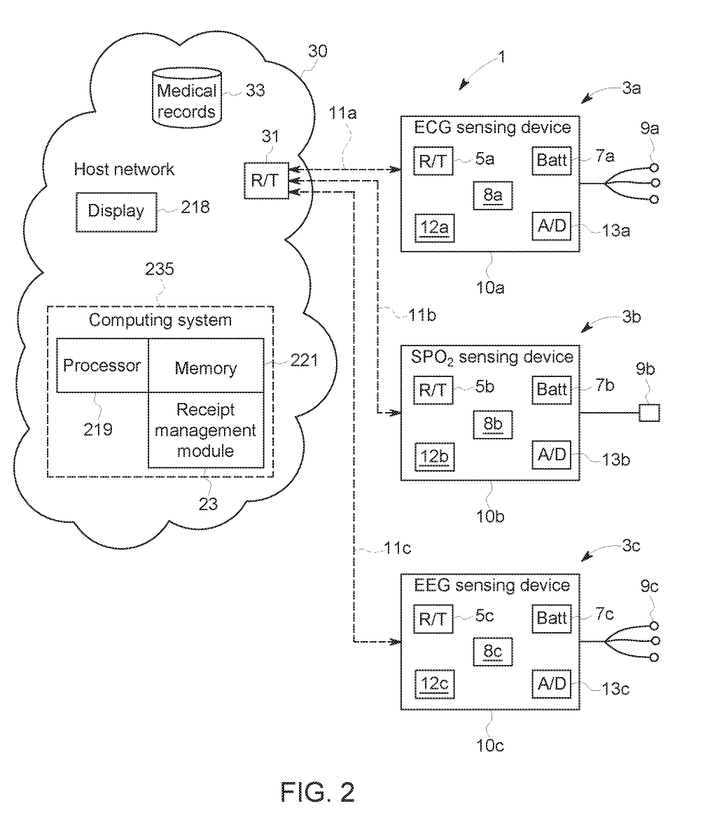

In still other embodiments, one or more of the sensing devices 3a-3c may transmit subset packets directly to the host network 30. In the embodiment of FIG. 2, the hub device 15 is omitted and the wireless sensing devices 3a-3c communicate directly with the host network 30. Thus, the receiver/transmitter 5a-5c of each wireless sensing device 3a-3c may communicate with a receiver/transmitter 31 associated with the host network 30 by the respective communication link 11a-11e. The communication link 11a-11e in this embodiment may operate according to any wireless communication protocol listed above. It may be desirable to operate the communication according to a wireless communication protocol that is appropriate for longer-range transmission. For example, the wireless sensing devices 3a-3c may communicate with the host network 30 on the WMTS spectrum or on the Wi-Fi spectrum. In such an embodiment, receiver/transmitters 31 may be provided throughout a patient care facility, such as a hospital, as needed based on the system configuration and the location of patients being monitored by wireless sensor devices. The host network 30 may house the computing system 235 containing the monitoring regulation module 23, and thus the calculation of the patient condition index and measurement interval assignment may be conducted by the computing system 235 housed in the host network 30. Further, the host network 30 may provide one or more central monitoring stations, such as user interfaces at central locations for attending clinicians to monitor patient conditions and/or receive alarm notifications.

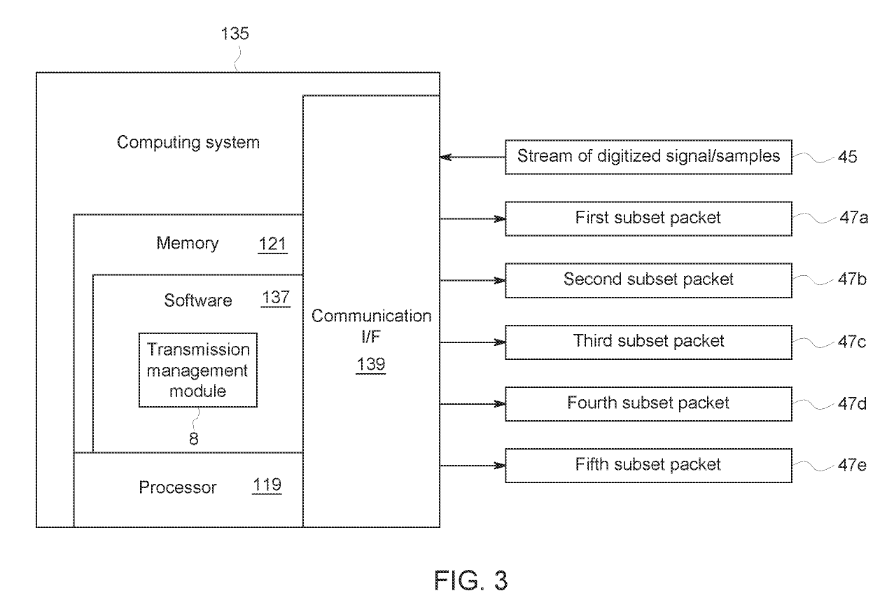

FIG. 3 provides a system diagram of a computing system 135 (e.g. each of the computing system 135a-135c in each sensing device 3a-3c) having a transmission management module 8 executable to divide the stream of digitized signal samples 45 into multiple interlaced subsets, which in the depicted embodiment includes 5 interlaced subsets, and to generate and transmit subset packets 47-47e accordingly. The computing system 135 includes a processor 119, memory 121, software 137, and communication interface 139. The processor 19 loads and executes software 37 from memory 21, including the transmission management module 8, which is an application within the software 37. Each transmission management module 8 includes computer-readable instructions that, when executed by the computing system 135 (including the processor 19), direct the operation as described in detail herein.

Although the computing system 135 as depicted in FIG. 3 includes one software element 137 encapsulating one transmission management module 8, it should be understood that one or more software elements having one or more modules may provide the same operation. Similarly, while the description provided herein refers to a single computing system 135 having a single processor 119, it is to be recognized that implementations of such systems can be performed using one or more processors, which may be communicatively connected, and such implementations are considered to be within the scope of the description. Likewise, the computing system 135 may be implemented as several computing systems networked together, including in a cloud computing environment. Such an embodiment may be utilized, for example, where the computing system 135 is implemented in a host network 30. It should also be noted that, while the transmission management module 8 is described herein as implemented on and by the sensing devices 3a-3c, in other embodiments the transmission management module 8 may be implemented by other devices within the system for secure, reliable, low power transmission (e.g. implemented by the hub 15 for transmission to the host network 30 or to another device in the system 1).

The memory 121, which includes the medical record database 133, can comprise any storage media, or group of storage media, readable by processor 119 and/or capable of storing software 137. The memory 121 can include volatile and non-volatile, removable and non-removable storage media implemented in any method or technology for storage of information, such as computer-readable instructions, data structures, program modules, or other data. Memory 121 can be implemented as a single storage device but may also be implemented across multiple storage devices or sub-systems. For example, the software 137 may be stored on a separate storage device than the medical record database 133. Further, in some embodiments the memory 121 may also store the medical record database 133, which could also be distributed, and/or implemented across one or more storage media or group of storage medias accessible within the host network 130. Similarly, medical record database 133 may encompass multiple different sub-databases at different storage locations and/or containing different information which may be stored in different formats.

Examples of memory devices, or storage media, include random access memory, read only memory, magnetic discs, optical discs, flash memory, virtual memory, and non-virtual memory, magnetic sets, magnetic tape, magnetic disc storage or other magnetic storage devices, or any other medium which can be used to storage the desired information and that may be accessed by an instruction execution system, as well as any combination or variation thereof, or any other type of storage medium. Likewise, the storage media may be housed locally with the processor 19, or may be distributed in one or more servers, which may be at multiple locations and networked, such as in cloud computing applications and systems. In some implementations, the store media can be a non-transitory storage media. In some implementations, at least a portion of the storage media may be transitory. Memory 121 may further include additional elements, such a controller capable, of communicating with the processor 119.

The communication interface 139 is configured to provide communication between the processor 19 and the various other aspects of the system 1, including the A/D converters 13a-13b to receive the stream of digitized signal samples 45 and to communicate with receiver/transmitters 5a-5c to transmit the subset packets 47a-47e to the hub 15 and/or host network 30.

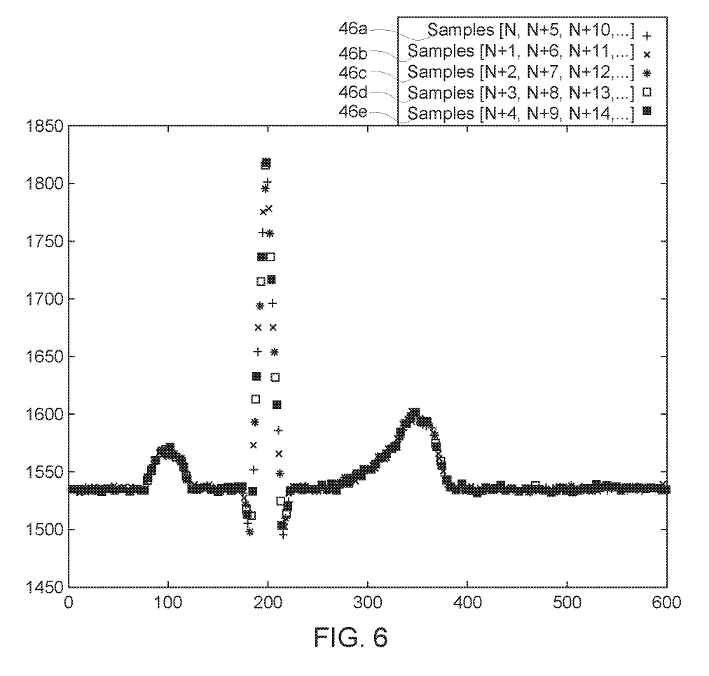

FIG. 5 depicts an exemplary physiological signal 41 that is an ECG, based upon which a stream of digitized samples 45 has been generated. FIG. 5 shows an exemplary time section of 1 second of the ECG signal, represented by 600 digitized signal samples (sample numbers signified along the x axis). The ECG includes a QRS wave 42a and T wave 42b, which are generally considered important aspects of the ECG physiological signal 41. FIG. 6 depicts the same digitized sample set of FIG. 5 divided into five exemplary interlaced subsets 46a-46e. Specifically, the digitized signal samples 45 of FIG. 4 are divided into a first interlace subset 46a including samples N, N+5, N+10, etc.; a second interlaced subset 46b including samples N+1, N+6, N+11, etc.; a third interlaced subset 46c including samples N+2, N+7, N+12, etc.; a fourth interlaced subset including samples N+3, N+8, N+13, etc.; and a fifth interlaced subset 46e including samples N+4, N+9, N+14, etc. FIG. 6 depicts the signal samples in each respective interlaced subset by a different symbol. As depicted, each interlaced subset 46a-46e is comprised of non-adjacent signal samples, which in the depicted embodiment are spread equidistantly in time from one another as every fifth sample (i.e. the sample interval between each non-adjacent signal sample in each interlaced subset is five samples).

In the example, each subset generally contains enough information to approximate the respective time section of the ECG signal. While each signal does not contain all important details of the ECG, such as a point describing each peak of the respective QRS wave, each subset provides enough information to roughly depict the ECG signal. In an instance where a data packet containing the signal samples for the QRS complex is lost, this method is superior to the prior art data transmissions methods described above because only a portion of the QRS complex data is lost. In the depicted embodiment, the remaining, received interlaced subsets can be utilized, and interpolation can be performed to depict the physiological signal as accurately as possible based on the available data.

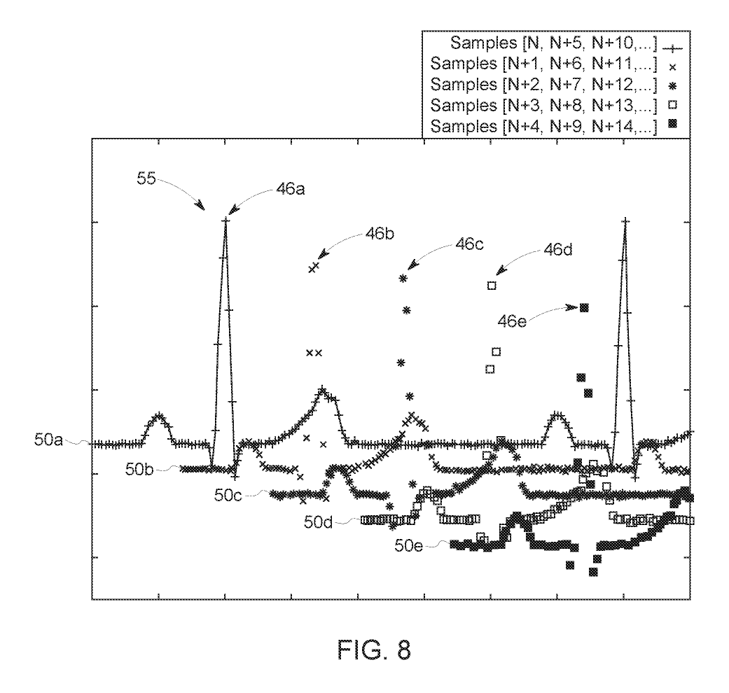

Each interlaced subset 46a-46e is transmitted in one or more subset packets 47a-47e. In certain embodiments, the interlaced subsets of signal samples may be compressed in order to reduce the size of each subset packet 47a-47e. The subset packets 47a-47e may each be transmitted on a separate transmission channel between the sensing device 3a-3c and the hub 15 or host network 30. In such an embodiment, the subset packets 47a-47e may be transmitted simultaneously or the transmission may be staggered in time in order to mask any information about the physiological data contained therein. In other embodiments, the subset packets 47a-47e may be transmitted over a single channel, and thus transmitted sequentially. In various sensing applications, multiple physiological signals or lead channels may be recorded simultaneously—e.g., for a twelve-lead ECG—and each physiological signal or lead is divided into interlaced subsets (e.g. 46a-46e) and transmitted as subset packets (e.g. 47a-47e) as described herein. Referring to FIG. 9, for example, three streams of digitized signal samples are received, one in each of the three depicted lead channels. Each stream of digitized signal samples is then divided into three sub-channels. Each sub-channel provides transmission of a respective subset packet containing one of the interlaced subsets of signal samples.

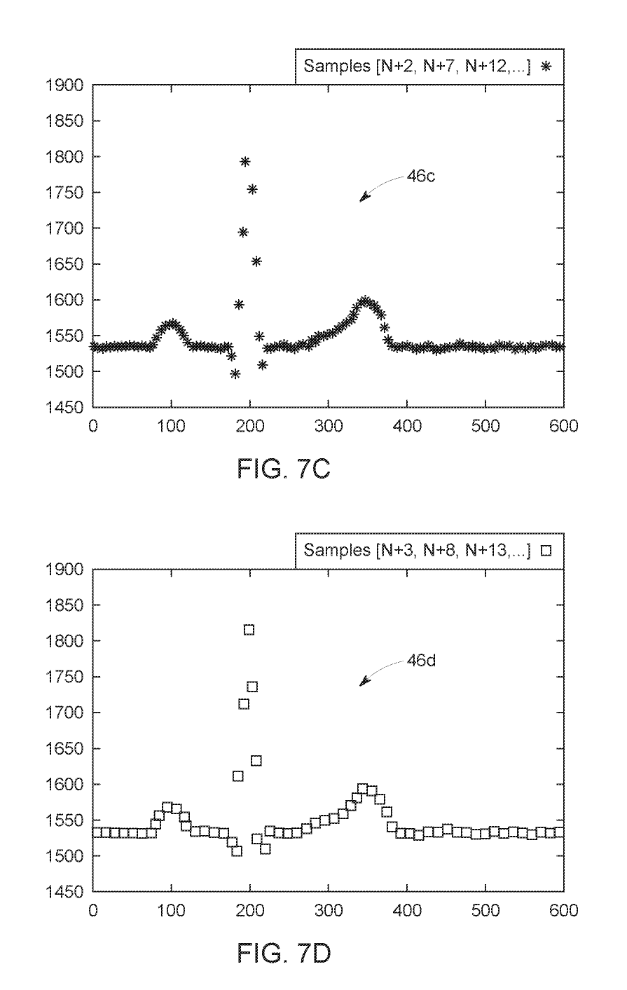

In certain embodiments, transmission of one or all of the subset packets may be delayed in order to mask any physiological signal aspects. Further, distributing the transmission start times can be utilized to more evenly distribute data packets, thereby using the radio bandwidth more effectively by distributing the data packets over time. FIG. 8 depicts one such embodiment, where the transmission start time for each subset packet is different. In the depicted embodiment, the start times are staggered by a constant and equal delay between each interlaced subset 46a-46e of about 225 milliseconds. The figure represents the time distribution of the transmission start times 50a-50e of each interlaced subset 46a-46e (which would be compressed and encoded in subset packets for transmission). The interlaced subsets are separated along the Y axis only to improve readability, and thus the Y axis values of sample points in FIG. 8 should be ignored. In the depicted example, the total transmission time is 900 milliseconds for sending all of the interlaced subsets for one time section of the ECG signal. In other embodiments, the total transmission time may be longer or shorter depending on the delay duration between the transmission start times.

While the delay duration between each start time 50a-50e is evenly distributed in the depicted embodiment, other embodiments may provide varied delay durations between each start time 50a-50e. In one embodiment, the delay between each start time 50a-50e may be a randomized value between a minimum delay amount and a maximum delay amount. This may improve the physiological signal masking even further by randomizing the delays between subsets so that no information can be gleaned based on the packet size of the subset packets 47a-47e.

In another embodiment, the size of the subset packets may be masked by varying the time section of digitized signal samples included in the respective interlaced subsets. Namely, the patterns of interlaced non-adjacent signal samples may start at different sample points (different N number points). As shown in FIG. 9, the interlaced subsets in each sub-channel start at a different sample point, and thus include different time sections of the stream of digitized signal samples. This can provide further data security in addition to the staggering via time delay, or may be an alternative to the delay in transmission start time.

The interlaced subsets may be created from the stream of digitized signal samples using any number of interlacing patterns, which may all start with respect to a single sample point or may be staggered as described above with respect to FIG. 9. FIGS. 6-8 demonstrate an interlacing pattern of every fifth sample point, and thus all of the interlacing subsets contain non-adjacent signal samples that are evenly distributed in time and separated by a sample interval of five samples. In other embodiments, the interlacing subsets may have varying patterns, such as varying intervals between non-adjacent signal samples between interlaced subsets, or even within a single interlaced subset. Furthermore, in certain embodiments one or more of the interlaced subsets may include adjacent and non-adjacent samples—i.e., series of adjacent samples separated by a sample interval. To provide just one example, an interlacing pattern of two interlaced subsets could be provided where a first interlaced subset includes samples N, N+1, N+4, N+5, etc.; and a second interlaced subset may include samples N+2, N+3, N+6, N+7, etc. In such an embodiment, each interlaced subset contains both adjacent and non-adjacent signal samples.

Additionally, various embodiments may be implemented where the stream of digitized signal samples may be divided into any number of two or more interlaced subsets. While examples are provided and depicted herein containing two, three, and five interlaced subsets, a person having ordinary skill in the art will understand that different applications of physiological signal transmission may be optimized by breaking the digitized signal into any number of two or more interlaced subsets for transmission.

The present inventor further recognized that in physiological patient monitoring, time delays between sensing data from a patient and displaying data to a clinician should be minimized as much as possible. Accordingly, the inventor recognized further potential benefit of the disclosed method in that the physiological signal can be approximated based on a single subset packet. Accordingly, information can be displayed to a clinician after the first subset packet (e.g. 47a) is received and processed at the receiving device. For example, the first subset packet 47a containing the first interlaced subset 46a can be interpolated to provide and display a first interpolated physiological signal section to approximate the physiological data for the clinician until updated and more precise information is available. FIG. 8 demonstrates this concept, where a first interpolated physiological signal section 55 is presented based on the non-adjacent signal samples in the first interlaced subset 46a. As can be seen from the graph in FIG. 6, the non-adjacent signal samples in the first interlaced subset 46a do not provide a perfect representation of the stream of digitized signal samples because, for example, they do not contain the Q peak or the R peak values of the digitized physiological signal. However, they enable an approximation of the stream of digitized data samples sufficient to present an initial graphical representation of the relevant time section of the physiological signal on a display. The first interpolated physiological signal section 55 can then be updated and replaced by an improved depiction as subsequent data becomes available.

For example, upon receiving a subsequent interlaced subset, a new interpolation can be performed based on the first interlaced subset 46a and whatever subsequent interlaced subsets are available (e.g. 47b-46e) in order to generate a subsequent interpolated physiological signal section. The display can then be updated to replace the first interpolated physiological signal section 55 with the most accurate available subsequent interpolated physiological signal section. The display can be finalized once all of the interlaced subsets (e.g. 46a-46e) are received. However, if one or more data packets are lost, at least an approximation of the relevant time section of the physiological signal can be provided based upon which the clinician can obtain information. In certain embodiments, an alert or notice may be provided along with the physiological signal to notify the clinician that one or more of the interlaced subsets (e.g. 46a-46e) have not been received and are not presented in the representation of the physiological signal.

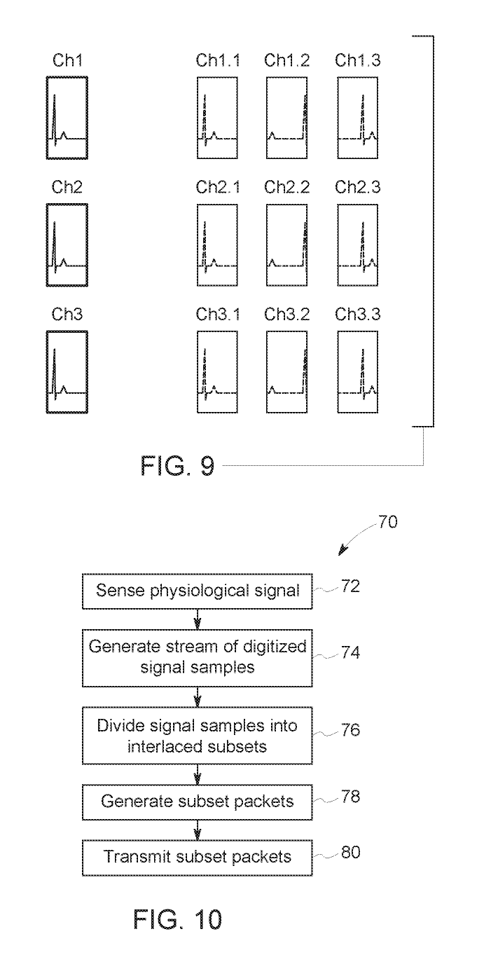

FIG. 10 depicts one embodiment of a method of patient monitoring including steps executed by a transmission management module to generate and transmit subset packets. A physiological signal is sensed at step 72 and a stream of digitized signal samples is generated at step 74 based on the sensed physiological signal. The stream of digitized signal samples is divided into two or more interlaced subsets at step 76 in accordance with one of the various embodiments described above. A subset packet is generated at step 78 for each interlaced subset, which may include compressing the signal samples in each interlaced subset and encoding the compressed data. The subset packet is transmitted at step 80.

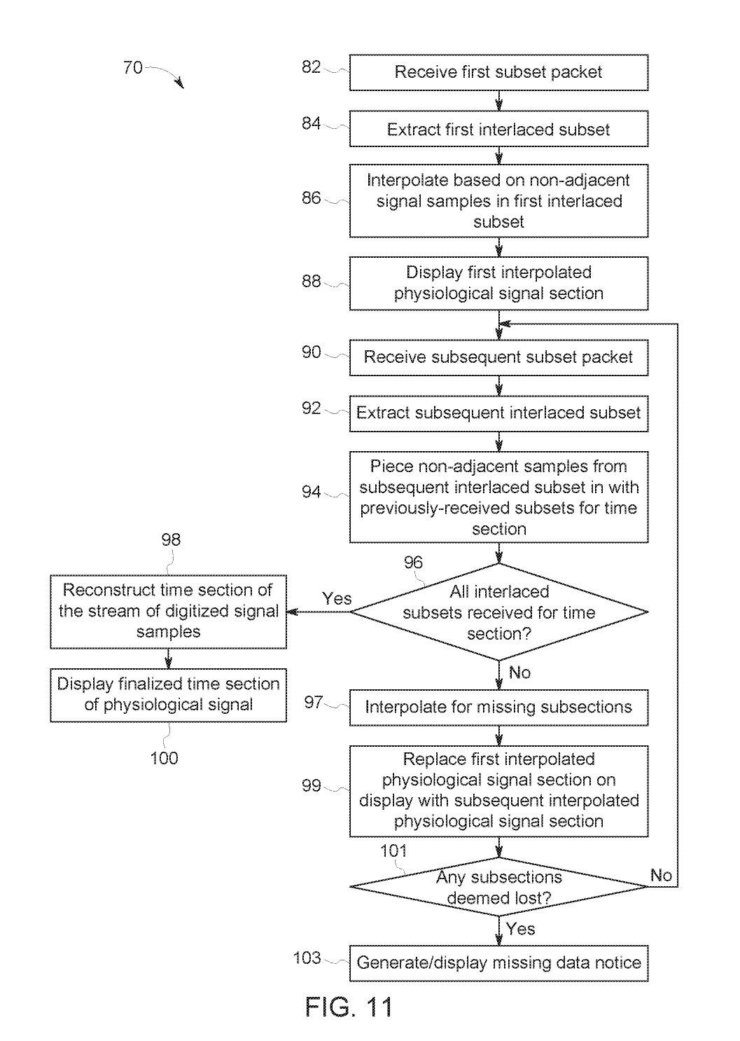

FIG. 11 depicts one embodiment of a method 70 of monitoring a patient, including steps executed by a receipt management module in receiving and displaying the data contained in the subset packets. A first subset packet is received at step 82, and the first interlaced subset data is extracted at step 84, such as by decrypting and decompressing the subset packet. Instructions are executed at step 86 to interpolate based on the non-adjacent signal samples in the first interlaced subset to generate a first interpolated physiological signal section. The first interpolated physiological signal section is displayed at step 88. Meanwhile, a subsequent subset packet is received at step 90 and the subsequent interlaced subset is extracted at step 92. At step 94, the non-adjacent samples from the subsequent interlaced subset are pieced together with the previously-received non-adjacent signal samples from previously received interlaced subsets for the respective time section of the physiological signal. Step 96 is executed to determine whether all interlaced subsets for the relevant time section have been received. If so, then the time section of the physiological signal is reconstructed at step 98 and the finalized reconstructed signal is displayed at step 100.

Returning to step 96, if all interlaced subsets have not been received, then the available data is interpolated to estimate the values of the missing data. The display is then updated to display the subsequent interpolated physiological signal section in place of the first interpolated physiological signal section. Step 101 is executed to determine whether any subsections are deemed lost—i.e. will not be received or recovered. For example, this may be a time-based analysis or based on the exhaustion of recovery steps executed by the respective transmitter/receiver devices and modules. If one or more subsections are deemed lost and unrecoverable, then a missing data notice is generated at step 103 to provide notice regarding the missing data, and hence the possible unreliability of the most recent subsequent interpolated physiological signal section. In another embodiment, the missing data notice may be generated and/or displayed along with the first interpolated physiological signal portion, and the missing data notice may continue to be displayed with each subsequent interpolated signal portion until all of the interlaced subset packets have been received for that time section.

This written description uses examples to disclose the invention, including the best mode, and also to enable any person skilled in the art to make and use the invention. Certain terms have been used for brevity, clarity and understanding. No unnecessary limitations are to be inferred therefrom beyond the requirement of the prior art because such terms are used for descriptive purposes only and are intended to be broadly construed. The patentable scope of the invention is defined by the claims, and may include other examples that occur to those skilled in the art. Such other examples are intended to be within the scope of the claims if they have features or structural elements that do not differ from the literal language of the claims, or if they include equivalent features or structural elements with insubstantial differences from the literal languages of the claims.

1. A wireless patient monitoring system comprising:
one or more sensing devices, each sensing device having:
a sensor that senses at least one physiological signal of a patient;
an analog-to-digital converter that generates a stream of digitized signal samples based on the physiological signal;
a first processor;
a transmission management module executable on the first processor to:
divide the stream of digitized signal samples into two or more interlaced subsets, wherein each interlaced subset contains non-adjacent signal samples from the stream of digitized signal samples;
generate at least one subset packet based on each of the two or more interlaced subsets;
control a wireless transmitter to wirelessly transmit the subset packets;
a receipt management module executable on a second processor to:
receive the subset packets from the one or more sensing devices;
extract each of the two or more interlaced subsets of non-adjacent signal samples from the subset packets; and
piece the non-adjacent signal samples in the interlaced subsets together to reconstruct the stream of digitized signal samples.

2. The wireless patient monitoring system of claim 1, wherein each of the interlaced subsets contains only non-adjacent signal samples from the time section.

3. The wireless patient monitoring system of claim 2, wherein the each of the non-adjacent signal samples in the interlaced subset is equidistant in time from one another.

4. The wireless patient monitoring system of claim 3, wherein the transmission management module is executable to divide a time section of the stream of digitized signal samples into three interlaced subsets, including a first interlaced subset containing at least a first, fourth, and seventh digitized signal sample in the time section, a second interlaced subset containing at least a second, fifth, and eighth digitized signal sample in the time section, and a third interlaced subset containing at least a third, sixth, and ninth digitized signal sample in the time section.

5. The wireless patient monitoring system of claim 1, wherein for at least one of the two or more interlaced subsets, a sample interval between the non-adjacent signal samples varies across that interlaced subset.

6. The wireless patient monitoring system of claim 1, wherein the transmission management module is executable to delay transmission for one or more of the subset packets by a delay duration with respect to a transmission of another one of the subset packets.

7. The wireless patient monitoring system of claim 6, wherein the transmission management module controls the wireless transmitter to transmit each subset packet at a different wireless transmission start time than the other subset packets.

8. The wireless patient monitoring system of claim 7, wherein the delay duration between each of the wireless transmission start times is equal.

9. The wireless patient monitoring system of claim 7, wherein the delay duration between each of the wireless transmission start times is a randomized value between a minimum delay and a maximum delay.

10. The wireless patient monitoring system of claim 1, the wherein the receipt management module is further executable to:
interpolate the non-adjacent signal samples from one or more of the interlaced subsets prior to receiving all of the interlaced subset packets for a time section of the stream of digitized signal samples to generate an interpolated physiological signal section; and
display the interpolated physiological signal section.

11. The wireless patient monitoring system of claim 10, the wherein the receipt management module is executable to:
upon receiving a first interlaced subset, interpolate the non-adjacent signal samples in the first interlaced subset to generate a first interpolated physiological signal section;
display the first interpolated physiological signal section on a display;
upon receiving a subsequent interlaced subset, interpolate the non-adjacent signal samples in the first interlaced subset and the subsequent interlaced subset to generate a subsequent interpolated physiological signal section; and
replace the first interpolated physiological signal section on the display with the subsequent interpolated physiological signal section.

12. A method of patient physiological monitoring, the method comprising:
sensing at least one physiological signal of a patient;
generating a stream of digitized signal samples based on the physiological signal;
dividing a time section of the stream of digitized signal samples into two or more interlaced subsets, wherein each interlaced subset contains non-adjacent signal samples from the time section of the stream of digitized signal samples;
generating at least one subset packet based on each of the two or more interlaced subsets;
wirelessly transmitting each of the subset packets;
receiving the wirelessly transmitted subset packets;
extracting each of the two or more interlaced subsets from the subset packets; and
piecing the non-adjacent signal samples in the interlaced subsets together to reconstruct the time section of the stream of digitized signal samples.

13. The method of claim 12, wherein each of the interlaced subsets contains only non-adjacent signal samples from the stream of digitized signal samples, and wherein each of the non-adjacent signal samples in the interlaced subset is equidistant in time from one another.

14. The method of claim 13, wherein the time section is divided into three interlaced subsets, including a first interlaced subset containing at least a first, fourth, and seventh digitized signal sample in the time section, a second interlaced subset containing at least a second, fifth, and eighth digitized signal sample in the time section, and a third interlaced subset containing at least a third, sixth, and ninth digitized signal sample in the time section.

15. The method of claim 12, wherein, within at least one of the two or more interlaced subsets, a sample interval between the non-adjacent signal samples varies across that interlaced subset.

16. The method of claim 12, further comprising delaying the wireless transmission of one or more of the subset packets by a delay duration with respect to a transmission time of another one of the subset packets.

17. The method of claim 16, wherein each subset packet has a different wireless transmission start time than the other subset packets.

18. The method of claim 17, wherein the delay duration between each of the wireless transmission start times is equal.

19. The method of claim 17, wherein the delay duration between each of the wireless transmission start times is a randomized value between a minimum delay and a maximum delay.

20. The method of claim 12, further comprising:
prior to receiving all of the interlaced subset packets for the time section, interpolating the non-adjacent signal samples in the first interlaced subset to generate a first interpolated physiological signal section;
displaying the first interpolated physiological signal section on a display;
upon receiving a subsequent interlaced subset, interpolating the non-adjacent signal samples in the first interlaced subset and the subsequent interlaced subset to generate a subsequent interpolated physiological signal section; and
replacing the first interpolated physiological signal section on the display with the subsequent interpolated physiological signal section.
